(19) United States
(12) Patent Application Publication
(10) Pub. No.: US 2022/0000727 A1
(43) Pub. Date: Jan. 6, 2022

(54) ORGANIC AND MINERAL FILTER HYBRID SUNSCREEN

(71) Applicant: L'OREAL, Paris (FR)

(72) Inventors: Jennifer Lynn PAULUCCI, Manalapan, NJ (US); Maximillian Pacifico BARIA, River Edge, NJ (US); Patricia BRIEVA, Manalapan, NJ (US); Jaimie MECCA, Clifton, NJ (US)

(73) Assignee: L'OREAL, Paris (FR)

(21) Appl. No.: 16/917,966

(22) Filed: Jul. 1, 2020

(51) Int. Cl. A61K 8/06; A61Q 17/04; A61Q 19/00; A61K 8/27; A61K 8/37; A61K 8/92

(52) U.S. Cl. CPC A61K 8/064; A61Q 17/04; A61Q 19/00; A61K 2800/10; A61K 8/37; A61K 8/92; A61K 8/27

(57) ABSTRACT

A sunscreen composition provided as a water-in-oil emulsion of aqueous and oil phases includes a combination of organic and mineral UV filters, and one or a combination of, each, of dispersing agents, film formers, emulsifiers, structuring agents, and aqueous and oil phase solvents. The sunscreen composition confers water-resistant broad-spectrum UV protection, is non-whitening, and demonstrates acceptable sensorial properties, and stability through a range of temperatures.

### FIELD OF THE INVENTION

The present disclosure is directed to skin care compositions that provide UV protection, in particular for protecting skin from ultraviolet (“UV”) radiation.

### BACKGROUND OF THE INVENTION

The photoprotection of keratinous substrates, including both skin and hair, is considered by many to be necessary in order to facilitate protection from sun-damage, sunburn, and photo-aging, as well as to decrease the chances of skin cancer development caused by exposure to UV radiation.

It is an object of the present disclosure to provide a sunscreen composition, in some particular embodiments, an emulsion sunscreen composition, that provides SPF properties employing a hybrid UV filter system that includes organic and mineral UV filters that remains stable under varied environmental conditions and is generally aesthetically pleasing upon application, particularly application to the skin.

Currently marketed sunscreen compositions suffer from the inclusion of certain UV filters that have been determined to be unfavorable for some purposes, in particular, but not limited to oxybenzone and octinoxate organic UV filters. Some sunscreens have reduced or eliminated less favored UV filters and have increased use of mineral based UV filters, unfortunate with a concomitant effect of causing whitening when applied to skin due to the features of the mineral based UV filters and the reagents employed to formulate them. Thus, there is a need for sunscreen that deliver high SPF protection that is water resistant and lacks certain of the challenges posed by disfavored organic UV filters, and high amounts of mineral UV filters.

### BRIEF SUMMARY OF THE INVENTION

In accordance with various embodiments, provided is a water-in-oil emulsion sunscreen composition comprising aqueous and oil phases that include a combination of organic and inorganic/mineral UV filters, and one or a combination of each of dispersing agents, film formers, emulsifiers, structuring agents, and aqueous and oil phase solvents. The sunscreen composition confers water-resistant broad-spectrum UV protection, is non-whitening, and demonstrates acceptable sensorial properties and emulsion structural stability through a range of temperatures.

In a representative embodiment, the sunscreen composition comprises: a water-in-oil emulsion of aqueous and oil phases, comprising the following components:
(i) at least one mineral UV filter, present from about 10% to about 14%;
(ii) a blend of organic UV filters, present from about 15% to about 45%;
(iii) at least one film former, present from about 2% to about 5%;
(iv) one or a combination of dispersing agents;
(v) one or a combination of emulsifiers;
(vi) one or a combination of structuring agents; and
(vii) one or a combination of solvents,
wherein the components are present by weight, based on the total weight of the sunscreen composition.

In one particular embodiment, the sunscreen composition comprises: a water-in-oil emulsion of aqueous and oil phases, comprising the following components:
(i) at least one mineral UV filter comprising ZnO, present at about 12%;
(ii) a blend of organic UV filters comprising homosalate, present at about 15%, octisalate, present at about 5%, and octocrylene, present at about 10%;
(iii) at least one film former comprising one or more of polyester-5 and dimethicone acrylates copolymer, present from about 2% to about 5%;
(iv) a combination of dispersing agents comprising polyhydroxystearic acid, and isohexadecane (and) disteardimonium hectorite (and) propylene carbonate;
(v) a combination of emulsifiers comprising PEG-30 dipolyhydroxystearate and polyglyceryl-4 isostearate;
(vi) one or a combination of structuring agents; and
(viii) one or a combination of solvents,
wherein the components are present by weight, based on the total weight of the sunscreen composition.

In some particular embodiments, the sunscreen composition is free from one or both of oxybenzone and octinoxate organic UV filters.

In some embodiments, the components of the aqueous phase and the components of the oil phase, respectively, are present in the sunscreen composition at a weight ratio of aqueous components to oil components in a range from about >1:1. In some embodiments, a phase ratio of the aqueous phase to the non-aqueous phase is calculated based on the total weight of the aqueous phase components to the weight of non-aqueous components of the oil phase.

In the various embodiments, the sunscreen composition comprises one or a combination of organic UV filters, and one or a combination of inorganic/mineral UV filters. In some particular embodiments, the organic UV filters include each of homosalate, octisalate and octocrylene, and the inorganic/mineral UV filters include zinc oxide.

In some embodiments of the sunscreen composition, the combination of organic and inorganic/mineral UV filters is present in the sunscreen composition in a total amount the range from about 15% to about 45% based on the total weight of the sunscreen composition.

In some embodiments of the sunscreen composition, the combination of organic and inorganic/mineral UV filters includes zinc oxide present in an amount in the range from about 10% to about 14%, and a blend of organic UV filters comprising homosalate present in an amount in the range from about 12% to about 20% octisalate present in an amount in the range from about 1% to about 7%, and octocrylene present in an amount in the range from about 7% to about 15%, each present by weight based on the total weight of the sunscreen composition. in some particular embodiments of the sunscreen composition, the combination of organic and inorganic/mineral UV filters includes zinc oxide present in an amount in the range from about 10% to about 12%, homosalate present at about 15% octisalate present at about 5%, and octocrylene present at about 10%, each present by weight based on the total weight of the sunscreen composition. In some more particular embodiments, zinc oxide is present at about at least 12% by weight.

In some embodiments, the at least one film former comprises one or more of polyester-5 and dimethicone acrylates crosspolymer. In some particular embodiments, the at least one film former comprises one or more of polyester-5, present from about 1% to about 3%, and dimethicone acrylates crosspolymer, present from about 2% to about 5%, and a combination thereof, each by weight, based on the total weight of the sunscreen composition.

In some particular embodiments, the sunscreen composition comprises only polyester-5. In some particular embodiments, the sunscreen composition comprises both polyester-5 and dimethicone acrylates crosspolymer.

In some particular embodiments, the at least one film former is not any one of Polyurethane-35, Trimethylsiloxysilicate, Ethylenediamine/Stearyl Dimer Dilinoleate Copolymer, or a combination thereof.

In some embodiments, the one or a combination of dispersing agents comprises Polyhydroxystearic Acid, Isohexadecane (and) Disteardimonium Hectorite (and) Propylene Carbonate, or a combination thereof. In some particular embodiments, one or more of the Polyhydroxystearic Acid is present from about 0.1% to about 2%, by weight, based on the total weight of the sunscreen composition, and the Isohexadecane (and) Disteardimonium Hectorite (and) Propylene Carbonate is present from about 1% to about 4%, by weight, based on the total weight of the sunscreen composition.

In some embodiments, the one or a combination of emulsifiers comprises PEG-30 Dipolyhydroxystearate, Polyglyceryl-4 Isostearate, or a combination thereof. In some particular embodiments, the one or a combination of emulsifiers comprises PEG-30 Dipolyhydroxystearate, Polyglyceryl-4 Isostearate, or a combination thereof, present from about 2% to about 5%, by weight, based on the total weight of the sunscreen composition.

In some particular embodiments, the one ora combination of emulsifiers is not any one of Poly C10-30 Alkyl Acrylate, Lauryl PEG-9 Polydimethysiloxyethyl Dimethicone, Glyceryl Stearate (and) PEG-100 Stearate or a combination thereof.

In some embodiments, the one or a combination of structuring agents comprises Cetearyl Alcohol, present from about 0.5% to about 5%, Copernicia Cerifera (Carnuba) Wax/Copernicia Cerifera Cera, present from about 0.5% to about 2%, or a combination thereof, each by weight, based on the total weight of the sunscreen composition.

In some particular embodiments, the one or a combination of structuring agents is not Microcrystalline Wax/Cera Microcristallina.

In some embodiments, the sunscreen composition also includes one or more SPF boosters, one or more sensorial fatty compounds or feel modifying powders, or one or more actives.

In the various embodiments, the sunscreen composition is SPF water resistant and confers broad-spectrum protection. In the various embodiments, the sunscreen composition confers consumer acceptable spreading and sensorial properties including non-whitening characteristics. In the various embodiments, the sunscreen composition is temperature stable with a water-in-oil emulsion structure that is homogenous upon agitation at the time of application. The sunscreen composition includes high amounts of UV filters to provide an SPF (in vitro) in a range from about 30 to about 55. In some embodiments, the sunscreen composition has an SPF of about 30, or of about 45, or of about 50.

The present invention is also directed to a method of inhibiting UV radiation damage to a keratinous substrate, in particular, skin, by applying the above-disclosed sunscreen composition onto a surface of the keratinous substrate.

### DETAILED DESCRIPTION OF THE INVENTION

The present disclosure provides a sunscreen composition with high UV protection, SPF water resistance and broad-spectrum protection with an SPF that can range from about 30 to about 55. The sunscreen composition employs a hybrid UV filter system that includes inorganic/mineral and organic UV filters, and includes film formers and dispersing agents to achieve water resistance, and emulsifiers, solvents and structuring agents that provide consumer acceptable spreading and sensorial properties including non-whitening characteristics. The sunscreen composition is temperature stable with a fluid or cream type water-in-oil emulsion structure that can, after a time, exhibit a slight separation on the surface, and is homogenous upon agitation at the time of application.

The term “keratinous tissue” includes, but is not limited to, skin, hair, and nails.

The term “water-in-oil emulsion” or “W/O emulsion” includes a water phase dispersed in an oil phase, where the oil phase is a continuous phase.

The term “Homogenous” means in reference to the composition as having the visual appearance of being substantially uniform throughout, i.e., visually lacks separation and pooling of fluid.

The term “sun protection factor” or “SPF” is a value expressed mathematically by the ratio of the irradiation time necessary to attain the erythemogenic threshold with the UV screening agent to the time necessary to attain the erythemogenic threshold without UV screening agent. SPF generally provides information about the skin's resistance to ultraviolet B (UVB) radiation from the sun. The SPF rating system has been developed to provide consumer guidance in selecting sunscreens. The sunscreen composition according to the present disclosure can be formulated to achieve a variety of different SPFs. For example, the sunscreen composition can have an in vitro or in vivo SPF of at least 30, 35, 40, 45, 50, or 55 or higher (or in a range between any of these values).

The term “critical wavelength” is an absorption spectrum of a sunscreen composition characterized by an index, namely a wavelength, where the integral of the spectral absorbance curve reached 90% of the integral from 290 nm to 400 nm. The critical wavelength is used to determine the breadth of UV protection. In some embodiments, the sunscreen composition has a critical wavelength that is at least 370 nm.

The terms “water-resistant broad-spectrum UV protection” and “SPF water resistant” and “WR” refer generally to the property of preserved UV protection, for example, stable retention of SPF, after exposure to water under predetermined water exposure times and conditions. A composition demonstrates SPF or UV protection water resistance when the SPF is Suitable water resistance is achieved if a composition demonstrates preserved SPF after being submerged in a base of water after 40 or 80 minutes. Thus, for example, a sunscreen composition may bear the designation WR if the sunscreen demonstrates an SPF that is not diminished by more than 10% after application to a substrate and water submersion for from about 40 minutes up to about 80 minutes.

The term “emulsion structural stability” means that an sunscreen composition exhibits an initially good aesthetic appearance, including lack of a grainy texture and no visually perceptible separation of phases and does not demonstrate appreciable pooling of fluid. In addition, a sunscreen composition retains stability during extended storage including storage up to 4 weeks or up to 8 weeks including exposure to temperature up to about 45 degrees C. For example, a sunscreen composition is considered to demonstrate emulsion structural stability if the composition does not exhibit signs of phase separation, and/or become inhomogeneous, in particular after exposure to about 45 degrees C. for about 4 to about 8 weeks wherein any inhomogeneity is readily resolved by shaking or agitation of the composition. In some embodiments of the sunscreen composition comprising powders, the sunscreen composition demonstrates emulsion structural stability wherein it does not exhibit signs of phase separation, and/or become inhomogeneous, in particular after exposure to an ambient temperature of about 5 degrees C., or about 25 degrees C., or about 37 degrees C., or about 45 degrees C. for up to about 8 weeks wherein any inhomogeneity is readily resolved by shaking or agitation of the composition. In contrast, an emulsion that does not have structural stability demonstrates inhomogeneity and phase separation either initially, or after exposure to ambient temperatures of about 45 degrees C. for about 4 to about 8 weeks, wherein the inhomogeneity is not readily resolved by shaking or agitation of the composition.

The term “SPF booster” refers to a material which increases the UV absorption of another material when the two are intermixed in a composition by refracting UV radiation, thereby increasing the effective path length of the UV radiation through the sunscreen composition.

In the various embodiments, the sunscreen composition includes
(i) at least one mineral UV filter, present from about 10% to about 14%;
(ii) a blend of organic UV filters, present from about 15% to about 45%;
(iii) at least one film former, present from about 2% to about 5%;
(iv) one or a combination of dispersing agents;
(v) one or a combination of emulsifiers;
(vi) one or a combination of structuring agents;
(vii) one or a combination of solvents; and
(viii) optionally, one or a combination of SPF boosters, one or a combination of sensorial fatty compounds or feel modifying powders, or one or a combination of actives.

UV Filters

In accordance with the various embodiments, the sunscreen composition according to the disclosure includes one or a combination of each of inorganic/mineral based and organic UV filters. The organic UV filters are typically present in the oil phase of the emulsion.

In some embodiments, the sunscreen composition includes zinc oxide and one or a combination of organic UV filters. In some embodiments, the organic UV filter may be selected from homosalate, octisalate (ethylhexyl salicylate), octocrylene and combinations of these. In some embodiments, the sunscreen compositions include each of octisalate, homosalate, octocrylene.

In some particular embodiments, the sunscreen composition excludes, or is free from or devoid of one or both of oxybenzone and octinoxate organic UV filters.

In some particular embodiments, the amount of an inorganic/mineral UV filter is present in the sunscreen composition in the range of from about 10% to about 14%, or from about 11% to about 13%, or at least about 10%, or at about 11%, or at about 12%, or any suitable combination, sub-combination, range, or sub-range thereof by weight, based on the total weight of the sunscreen composition. In some particular embodiments, the UV filter system includes an inorganic UV filter comprising zinc oxide present in an amount that is at least about 10% wherein the composition can achieve a critical wavelength of about 370 nm. In some particular embodiments an inorganic UV filter comprising zinc oxide is present in an amount that is at least about 12%. In accordance with the various embodiments, the inorganic UV filter, for example, zinc oxide, is present in an amount that is less than about 15%, and in some embodiments the inorganic UV filter is present in an amount that is less than about 14%, or less than about 13%, or not more than about 12%, or not more than about 11%, or not more than about 10% by weight based on the total weight of the sunscreen composition.

Thus, any one inorganic UV filter may be present, by weight, based on the total weight of the sunscreen composition, from about 10, 11, 12, 13, to about 14 weight percent, including increments and ranges therein and there between.

In some embodiments, the organic UV filters include homosalate, present in an amount in the range from about 12% to about 20%, octisalate, present in an amount in the range from about 1% to about 7%, and octocrylene, present in an amount in the range from about 7% to about 15%, each present by weight, based on the total weight of the sunscreen composition

Thus, an organic UV filter may be present in the sunscreen composition, by weight, based on the total weight of the sunscreen composition, from about 1, 2, 3, 4, 5, 6, 7, 8, 9, 10, 11, 12, 13, 14, 15, 16, 17, 18, 19 to about 20 weight percent, including increments and ranges therein and there between.

In some particular embodiments, the inorganic UV filters include zinc oxide, present in an amount in the range from about 10% to about 14%, and the organic UV filters include homosalate, present in an amount in the range from about 12% to about 20% or from about 12% to about 15%; octisalate, present in an amount in the range from about 1% to about 7% or from about 3% to about 5%; and octocrylene, present in an amount in the range from about 7% to about 15% or from about 7% to about 10%, each present by weight, based on the total weight of the sunscreen composition.

In some particular embodiments, the inorganic UV filters include zinc oxide, present in an amount in the range from about 10% to about 12%, and the organic UV filters include homosalate, present at up to about 15%, octisalate, present at up to about 5%, and octocrylene, present at up to about 10%, each present, by weight, based on the total weight of the sunscreen composition.

In accordance with the various embodiments, the total amount of UV filters inclusive of inorganic and organic filters present in the systems and compositions can range from about 15% to about 45%, or from about 20% to about 40%, or from about 20% to about 35%, or from about 20% to about 30%, or from about 20% to about 25%, or any suitable combination, sub-combination, range, or sub-range thereof by weight, based on the total weight of the sunscreen composition. In some particular embodiments, the UV filters are present from at least about 20%, or from at least about 21%, or from at least about 22%, or from at least about 23%, or from at least about 24%, or from at least about 25%, or from at least about 26%, or from at least about 27%, or from at least about 28%, or from at least about 29%, or from at least about 30% by weight based on the total weight of the sunscreen composition. One of ordinary skill in the art, however, will appreciate that other ranges are within the scope of the invention.

Thus, the combination of UV filters present, by weight, based on the total weight of the sunscreen composition, is from about 15, 16, 17, 18, 19, 20, 21, 22, 23, 24, 25, 26, 27, 28, 29, 30, 31, 32, 33, 34, 35, 36, 37, 38, 39, 40, 41, 42, 43, 44 to about 45 weight percent, including increments and ranges therein and there between.

In some particular embodiments, the sunscreen composition includes organic filters present at a maximum amount of about 15% homosalate, 5% octisalate, and 10% octocrylene, and inorganic sunscreens (zinc oxide and titanium dioxide) up to about 25% (each), all by weight based on the total weight of the sunscreen composition.

In some embodiments of the sunscreen composition, the weight ratio of organic:inorganic/mineral UV filters is in the range from about 2:1 to about 4:1, based on the total weight of the sunscreen composition. In some particular embodiments of the sunscreen composition, the weight ratio of organic:inorganic/mineral UV filters is in the range from about 2.5:1 to about 3:1, based on the total weight of the sunscreen composition.

Generally, UV filters, active in UV-A and/or UV-B regions, used for the present invention can be water-soluble, fat-soluble or insoluble in commonly used cosmetic solvents. In some exemplary embodiments, the UV filters are selected from oil soluble UV filters. UV-A filters comprise groups of compounds which absorb light predominantly in the range of wavelengths from 320 nm to 400 nm (UV-A), and UV-B filters comprise groups of compounds which absorb light predominantly in the range of wavelengths from 290 nm to 320 nm (UV-B). Examples of suitable UV filters include, may be water-soluble, fat-soluble or insoluble in commonly used cosmetic solvents, and may be inorganic or organic.

Inorganic/Mineral UV Filters

In various embodiments, the sunscreen composition comprises one or a combination of inorganic UV filters. The inorganic UV filter used for the present disclosure may be active in the UV-A and/or UV-B region. The inorganic UV filter may be hydrophilic and/or lipophilic. The inorganic UV filter is, in some embodiments, insoluble in solvents, such as water, and ethanol commonly used in cosmetics.

The inorganic UV filter be in the form of a fine particle such that the mean (primary) particle diameter thereof ranges from 1 nm to 50 nm, and in some embodiments 5 nm to 40 nm, and in some embodiments 10 nm to 30 nm. The mean (primary) particle size or mean (primary) particle diameter herein is an arithmetic mean diameter. In some embodiments, a particle may have a diameter that is has a mean particle size that is about or less than about 1 micron, or that is about or less than about 200 nm, or that is about or less than about 100 nm (sometimes referred to nanoscale). It will be understood by one of ordinary skill in the art that a particle that is referred to as nano scale for purposes of cosmetic applications including for the sunscreen composition according to this disclosure will have a mean particle size that is less than about 100 nm unless specifically identified otherwise, for example as in the case of nano zinc oxide which is sometimes referred to as nanoscale though the UV agent has a mean primary particle size that is less than 200 nm.

The inorganic UV filter can be selected from the group consisting of silicon carbide, metal oxides, which may or may not be coated, and mixtures thereof. And in some embodiments, the inorganic UV filters are selected from pigments formed of metal oxides, such as, for example, pigments formed of titanium oxide (amorphous or crystalline in the rutile and/or anatase form), iron oxide, zinc oxide, zirconium oxide, or cerium oxide, which are all UV photoprotective agents that are well known per se.

The inorganic UV filter may or may not be coated. The inorganic UV filter may have one or more coating. The coating may comprise one or more compound selected from the group consisting of alumina, silica, aluminum hydroxide, silicones, silanes, fatty acids or salts thereof (such as sodium, potassium, zinc, iron, or aluminum salts), fatty alcohols, lecithin, amino acids, polysaccharides, proteins, alkanolamines, waxes, such as beeswax, (meth)acrylic polymers, organic UV filters, and (per)fluoro compounds. It is in some embodiments desirable for the coating to include one or a combination of organic UV filter.

Of course, the inorganic UV filter made of metal oxides may, before their treatment with silicones, have been treated with other surfacing agents, in particular, with cerium oxide, alumina, silica, aluminum compounds, silicon compounds, or their mixtures. The coated inorganic UV filter may have been prepared by subjecting the inorganic UV filter to one or more surface treatments of a chemical, electronic, mechano-chemical, and/or mechanical nature with any of the compounds as described above, as well as polyethylenes, metal alkoxides (titanium or aluminum alkoxides), metal oxides, sodium hexametaphosphate, and those shown, for example, in Cosmetics & Toiletries, February 1990, Vol. 105, pp. 53-64.

The coated inorganic UV filter may be titanium oxides coated: with silica, such as the product “Sun veil” from Ikeda, and “Sunsil TIN 50” from Sunjin Chemical; with silica and with iron oxide, such as the product “Sunveil F” from Ikeda; with silica and with alumina, such as the products “Microtitanium Dioxide MT 500 SA” from Tayca, “Tioveil” from Tioxide, and “Mirasun TiW 60” from Rhodia; with alumina, such as the products “Tipaque TTO-55 (B)” and “Tipaque TTO-55 (A)” from Ishihara, and “UVT 14/4” from Kemira; with alumina and with aluminum stearate, such as the product “Microtitanium Dioxide MT 100 T, MT 100 TX, MT 100 Z or MT-01” from Tayca, the products “Solaveil CT-10 W” and “Solaveil CT 100” from Uniqema, and the product “Eusolex T-AVO” from Merck; with alumina and with aluminum laurate, such as the product “Microtitanium Dioxide MT 100 S” from Tayca; with iron oxide and with iron stearate, such as the product “Microtitanium Dioxide MT 100 F” from Tayca; with zinc oxide and with zinc stearate, such as the product “BR351” from Tayca; with silica and with alumina and treated with a silicone, such as the products “Microtitanium Dioxide MT 600 SAS”, “Microtitanium Dioxide MT 500 SAS”, and “Microtitanium Dioxide MT 100 SAS” from Tayca; with silica, with alumina and with aluminum stearate and treated with a silicone, such as the product “STT-30-DS” from Titan Kogyo; with silica and treated with a silicone, such as the product “UV-Titan X 195” from Kemira; with alumina and treated with a silicone, such as the products “Tipaque TTO-55 (S)” from Ishihara or “UV Titan M 262” from Kemira; with triethanolamine, such as the product “STT-65-S” from Titan Kogyo; with stearic acid, such as the product “Tipaque TTO-55 (C)” from Ishihara; or with sodium hexametaphosphate, such as the product “Microtitanium Dioxide MT 150 W” from Tayca. Other titanium oxide pigments treated with a silicone are, and in some embodiments TiO2 treated with octyltrimethylsilane and for which the mean size of the individual particles is from 25 and 40 nm, such as that marketed under the trademark “T 805” by Degussa Silices, TiO2 treated with a polydimethylsiloxane and for which the mean size of the individual particles is 21 nm, such as that marketed under the trademark “70250 Cardre UF TiO2Si3” by Cardre, and anatase/rutile TiO2 treated with a polydimethylhydrosiloxane and for which the mean size of the individual particles is 25 nm, such as that marketed under the trademark “Microtitanium Dioxide USP Grade Hydrophobic” by Color Techniques.

And in some embodiments, the following coated TiO2 can be used as the coated inorganic UV filter: Stearic acid (and) Aluminum Hydroxide (and) TiO2, such as the product “MT-100 TV” from Tayca, with a mean primary particle diameter of 15 nm; Dimethicone (and) Stearic Acid (and) Aluminum Hydroxide (and) TiO2, such as the product “S A-TTO-S4” from Miyoshi Kasei, with a mean primary particle diameter of 15 nm; Silica (and) TiO2, such as the product “MT-100 WP” from Tayca, with a mean primary particle diameter of 15 nm; Dimethicone (and) Silica (and) Aluminum Hydroxide (and) TiO2, such as the product “MT-Y02” and “MT-Y-110 M3S” from Tayca, with a mean primary particle diameter of 10 nm; Dimethicone (and) Aluminum Hydroxide (and) TiO2, such as the product “SA-TTO-S3” from Miyoshi Kasei, with a mean primary particle diameter of 15 nm; Dimethicone (and) Alumina (and) TiO2, such as the product “UV TITAN MI 70” from Sachtleben, with a mean primary particle diameter of 15 nm; and Silica (and) Aluminum Hydroxide (and) Alginic Acid (and) TiO2, such as the product “MT-100 AQ” from Tayca, with a mean primary particle diameter of 15 nm. In terms of UV filtering ability, TiO2 coated with one or a combination of organic UV filter is more desirable. For example, Avobenzone (and) Stearic Acid (and) Aluminum Hydroxide (and) TiO2, such as the product “HXMT-100ZA” from Tayca, with a mean primary particle diameter of 15 nm, can be used.

The uncoated titanium oxide pigments are, for example, marketed by Tayca under the trademarks “Microtitanium Dioxide MT500B” or “Microtitanium Dioxide MT600B”, by Degussa under the trademark “P 25”, by Wacker under the trademark “Oxyde de titane transparent PW”, by Miyoshi Kasei under the trademark “UFTR”, by Tomen under the trademark “ITS” and by Tioxide under the trademark “Tioveil AQ”. The uncoated zinc oxide pigments are, for example: those marketed under the trademark “Z-cote” by Sunsmart; those marketed under the trademark “Nanox” by Elementis; and those marketed under the trademark “Nanogard WOO 2025” by Nanophase Technologies. The coated zinc oxide pigments are, for example: those marketed under the trademark “Oxide Zinc CS-5” by Toshiba (ZnO coated with polymethylhydrosiloxane); those marketed under the trademark “Nanogard Zinc Oxide FN” by Nanophase Technologies (as a 40% dispersion in Finsolv TN, C12-C15 alkyl benzoate); those marketed under the trademark “Daitopersion Zn-30” and “Daitopersion Zn-50” by Daito (dispersions in oxyethylenated polydimethylsiloxane/cyclopolymethylsiloxane comprising 30% or 50% of zinc nano-oxides coated with silica and polymethylhydrosiloxane); those marketed under the trademark “NFD Ultrafine ZnO” by Daikin (ZnO coated with phosphate of perfiuoroalkyl and a copolymer based on perfluoroalkylethyl as a dispersion in cyclopentasiloxane); those marketed under the trademark “SPD-Z1” by Shin-Etsu (ZnO coated with a silicone-grafted acrylic polymer dispersed in cyclodimethylsiloxane); those marketed under the trademark “Escalol Z100” by ISP (alumina-treated ZnO dispersed in an ethylhexyl methoxycinnamate/PVP-hexadecene copolymer/methicone mixture); those marketed under the trademark “Fuji ZnO-SMS-10” by Fuji Pigment (ZnO coated with silica and polymethylsilsesquioxane); and those marketed under the trademark “Nanox Gel TN” by Elementis (ZnO dispersed at 55% in C12-C15 alkyl benzoate with hydroxystearic acid polycondensate). The uncoated cerium oxide pigments are marketed, for example, under the trademark “Colloidal Cerium Oxide” by Rhone-Poulenc.

The uncoated iron oxide pigments are, for example, marketed by Arnaud under the trademarks “Nanogard WCD 2002 (FE 45B)”, “Nanogard Iron FE 45 BL AQ”, “Nanogard FE 45R AQ”, and “Nanogard WCD 2006 (FE 45R)”, or by Mitsubishi under the trademark “TY-220”. The coated iron oxide pigments are, for example, marketed by Arnaud under the trademarks “Nanogard WCD 2008 (FE 45B FN)”, “Nanogard WCD 2009 (FE 45B 556)”, “Nanogard FE 45 BL 345”, and “Nanogard FE 45 BL”, or by BASF under the trademark “Oxyde de fer transparent”.

Organic UV Filters

In various embodiments, the sunscreen composition comprises one or a combination of organic UV filters. The organic UV filters may be active in the UV-A and/or UV-B region. The organic UV filter may be hydrophilic and/or lipophilic. The organic UV filter may be solid or liquid. The terms “solid” and “liquid” mean solid and liquid, respectively, at 25° C. under 1 atm.

The organic UV filter can be selected from the group consisting of anthranilic compounds; dibenzoylmethane compounds; cinnamic compounds; salicylic compounds; camphor compounds; benzophenone compounds; ÿ,ÿ-diphenylacrylate compounds; triazine compounds; benzotriazole compounds; benzalmalonate compounds; benzimidazole compounds; imidazoline compounds; bis-benzoazolyl compounds; p-aminobenzoic acid (PABA) compounds; methylenebis(hydroxyphenylbenzotriazole) compounds; benzoxazole compounds; screening polymers and screening silicones; dimers derived from a-alkylstyrene; 4,4-diarylbutadienes compounds; guaiazulene and derivatives thereof; rutin and derivatives thereof; flavonoids; bioflavonoids; oryzanol and derivatives thereof; quinic acid and derivatives thereof; phenols; retinol; cysteine; aromatic amino acids; peptides having an aromatic amino acid residue; and mixtures thereof.

Mention may be made, as examples of the organic UV filter(s), of those denoted below under their INCI names, and mixtures thereof. Anthranilic compounds: Menthyl anthranilate, marketed under the trademark “Neo Heliopan MA” by Haarmann and Reimer. Dibenzoylmethane compounds: Butyl methoxydibenzoylmethane, marketed, in particular, under the trademark “Parsol 1789” by Hoffmann-La Roche; and isopropyl dibenzoylmethane. Cinnamic compounds: Ethylhexyl methoxycinnamate, marketed, in particular, under the trademark “Parsol MCX” by Hoffmann-La Roche; isopropyl methoxycinnamate; isopropoxy methoxycinnamate; isoamyl methoxycinnamate, marketed under the trademark “Neo Heliopan E 1000” by Haarmann and Reimer; cinoxate (2-ethoxyethyl-4-methoxy cinnamate); DEA methoxycinnamate; diisopropyl methylcinnamate; and glyceryl ethylhexanoate dimethoxycinnamate. Salicylic compounds: Homosalate (homomentyl salicylate), marketed under the trademark “Eusolex HMS” by Rona/EM Industries; ethylhexyl salicylate, marketed under the trademark “Neo Heliopan OS” by Haarmann and Reimer; glycol salicylate; butyloctyl salicylate; phenyl salicylate; dipropyleneglycol salicylate, marketed under the trademark “Dipsal” by Scher; and TEA salicylate, marketed under the trademark “Neo Heliopan TS” by Haarmann and Reimer. Camphor compounds, in particular, benzylidenecamphor derivatives: 3-benzylidene camphor, manufactured under the trademark “Mexoryl SD” by Chimex; 4-methylbenzylidene camphor, marketed under the trademark “Eusolex 6300” by Merck; benzylidene camphor sulfonic acid, manufactured under the trademark “Mexoryl SL” by Chimex; camphor benzalkonium methosulfate, manufactured under the trademark “Mexoryl SO” by Chimex; terephthalylidene dicamphor sulfonic acid, manufactured under the trademark “Mexoryl SX” by Chimex; and polyacrylamidomethyl benzylidene camphor, manufactured under the trademark “Mexoryl SW” by Chimex. Benzophenone compounds: Benzophenone-1 (2,4-dihydroxybenzophenone), marketed under the trademark “Uvinul 400” by BASF; benzophenone-2 (Tetrahydroxybenzophenone), marketed under the trademark “Uvinul D50” by BASF; Benzophenone-3 (2-hydroxy-4-methoxybenzophenone) or oxybenzone, marketed under the trademark “Uvinul M40” by BASF; benzophenone-4 (hydroxymethoxy benzophonene sulfonic acid), marketed under the trademark “Uvinul MS40” by BASF; benzophenone-5 (Sodium hydroxymethoxy benzophenone Sulfonate); benzophenone-6 (dihydroxy dimethoxy benzophenone); marketed under the trademark “Helisorb 11” by Norway; benzophenone-8, marketed under the trademark “Spectra-Sorb UV-24” by American Cyanamid; benzophenone-9 (Disodium dihydroxy dimethoxy benzophenonedisulfonate), marketed under the trademark “Uvinul DS-49” by BASF; and benzophenone-12, and n-hexyl 2-(4-diethylamino-2-hydroxybenzoyl)benzoate (UVINUL A+ by BASF). ÿ,ÿ-Diphenylacrylate compounds: Octocrylene, marketed in particular under the trademark “Uvinul N539” by BASF; and Etocrylene, marketed in particular under the trademark “Uvinul N35” by BASF. Triazine compounds: Diethylhexyl butamido triazone, marketed under the trademark “Uvasorb HEB“ ” by Sigma 3V; 2,4,6-tris(dineopentyl 4″-aminobenzalmalonate)-s-triazine, bis-ethylhexyloxyphenol methoxyphenyl triazine marketed under the trademark «TINOSORB S» by CIBA GEIGY, and ethylhexyl triazone marketed under the trademark «UVTNUL T150» by BASF. Benzotriazole compounds, in particular, phenylbenzotriazole derivatives: 2-(2H-benzotriazole-2-yl)-6-dodecyl-4-methylpheno, branched and linear; and those described in U.S. Pat. No. 5,240,975. Benzalmalonate compounds: Dineopentyl 4′-methoxybenzalmalonate, and polyorganosiloxane comprising benzalmalonate functional groups, such as polysilicone-15, marketed under the trademark “Parsol SLX” by Hoffmann-LaRoche. Benzimidazole compounds, in particular, phenylbenzimidazole derivatives: Phenylbenzimidazole sulfonic acid, marketed in particular under the trademark “Eusolex 232” by Merck, and disodium phenyl dibenzimidazole tetrasulfonate, marketed under the trademark “Neo Heliopan AP” by Haarmann and Reimer. Imidazoline compounds: Ethylhexyl dimethoxybenzylidene dioxoimidazoline propionate. Bis-benzoazolyl compounds: The derivatives as described in EP-669,323 and U.S. Pat. No. 2,463,264. Para-aminobenzoic acid compounds: PABA (p-aminobenzoic acid), ethyl PABA, Ethyl dihydroxypropyl PABA, pentyl dimethyl PABA, ethylhexyl dimethyl PABA, marketed, in particular, under the trademark “Escalol 507” by ISP, glyceryl PABA, and PEG-25 PABA, marketed under the trademark “Uvinul P25” by BASF. Methylene bis-(hydroxyphenylbenzotriazol) compounds, such as 2,2′-methylenebis[6-(2H-benzotriazol-2-yl)-4-methyl-phenol] marketed in the solid form under the trademark “Mixxim BB/200” by Fairmount Chemical, 2,2′-methylenebis[6-(2H-benzotriazol-2-yl)-4-(1,1,3,3-tetramethylbutyl)phenol] marketed in the micronized form in aqueous dispersion under the trademark “Tinosorb M” by BASF, or under the trademark “Mixxim BB/100” by Fairmount Chemical, and the derivatives as described in U.S. Pat. Nos. 5,237,071 and 5,166,355, GB-2,303,549, DE-197,26,184, and EP-893,119, and Drometrizole trisiloxane, marketed under the trademark “Silatrizole” by Rhodia Chimie or—“Mexoryl XL” by L′Oreal. Benzoxazole compounds: 2,4-bis[5-1(dimethylpropyl)benzoxazol-2-yl-(4-phenyl)imino]-6-(2-ethylhexyl)imino-I,3,5-triazine, marketed under the trademark of Uvasorb K2A by Sigma 3V. Screening polymers and screening silicones: The silicones described in WO 93/04665. Dimers derived from a-alkylstyrene: The dimers described in DE-19855649. 4,4-Diarylbutadiene compounds: I,I-dicarboxy(2,2′-dimethylpropyl)-4,4-diphenylbutadiene.

In some embodiments the organic UV filter(s) may be selected from the group consisting of: butyl methoxydibenzoylmethane, ethylhexyl methoxycinnamate, homosalate, ethylhexyl salicylate, phenylbenzimidazole sulfonic acid, benzophenone-3, benzophenone-4, benzophenone-5, n-hexyl 2-(4-diethylamino-2-hydroxybenzoyl)benzoate, I, r-(I,4-piperazinediyl)bis[I-[2-[4-(diethylamino)-2-hydroxybenzoyl]phenyl]-methanone 4-methylbenzylidene camphor, terephthalylidene dicamphor sulfonic acid, disodium phenyl dibenzimidazole tetrasulfonate, ethylhexyl triazone, bis-ethylhexyloxyphenol methoxyphenyl triazine, diethylhexyl butamido triazone, 2,4,6-tris(dineopentyl 4′-aminobenzalmalonate)-s-triazine, 2,4,6-tris(diisobutyl 4′-aminobenzalmalonate)-s-triazine, 2,4-bis-(n-butyl 4′-aminobenzalmalonate)-6-[(3-{1,3,3,3-tetramethyl-1-[(trimethylsilyloxy]-disiloxanyl}propyl)amino]-s-triazine, 2,4,6-tris-(di-phenyl)-triazine, 2,4,6-tris-(ter-phenyl)-triazine, methylene bis-benzotriazolyl tetramethylbutylphenol, drometrizole trisiloxane, polysilicone-15, dineopentyl 4′-methoxybenzalmalonate, I,I-dicarboxy(2,2′-dimethylpropyl)-4,4-diphenylbutadiene, 2,4-bis[5-1 (dimethylpropyl)benzoxazol-2-yl-(4-phenyl)imino]-6-(2-ethylhexyl)imino-1,3,5-triazine, camphor benzylkonium methosulfate, and mixtures thereof.

Film Formers

In accordance with the various embodiments, the sunscreen composition according to the disclosure includes one or a combination of film formers.

In some embodiments, film formers that may be used in the sunscreen composition are selected from polyester sulfonate copolymers and silicone solutions. In some particular embodiments, the one or a combination of film formers comprises one or more of polyester-5, present from about 1% to about 3%, and dimethicone acrylates copolymer, present from about 2% to about 5%, each by weight, based on the total weight of the sunscreen composition. In some particular embodiments, the one or a combination of film formers is not any one of Polyurethane-35, Trimethylsiloxysilicate, Ethylenediamine/Stearyl Dimer Dilinoleate Copolymer, Acrylates/Polytrimethylsiloxymethacrylate Copolymer, Dimethicone (and) Dimethiconol, PolyMethylsilsesquioxane, VP/Eicosene Copolymer, Acrylates Copolymer, Polyurethane-6, Polyurethane-34, VP/Hexadecene Copolymer or a combination thereof. It will be appreciated that in embodiments wherein the film former is not selected from one of the foregoing film formers, the sunscreen will be devoid of or will exclude or will be essentially free from one or more of Polyurethane-35, Trimethylsiloxysilicate, Ethylenediamine/Stearyl Dimer Dilinoleate Copolymer, Acrylates/Polytrimethylsiloxymethacrylate Copolymer, Dimethicone (and) Dimethiconol, PolyMethylsilsesquioxane, VP/Eicosene Copolymer, Acrylates Copolymer, Polyurethane-6, Polyurethane-34, and VP/Hexadecene Copolymer. These excluded film formers can be associated with one or more undesirable properties that include: unpleasant and noticeable residue on skin; incompatibility with the sunscreen composition system; and causes instability and separation over time, for example at 4 weeks which separation cannot be overcome by agitations/shaking of the composition.

Each one or a combination of film formers is present in the sunscreen composition at a concentration from about 0.01% to 15%, in some embodiments from about 0.1% to 10%, and in some embodiments from about 0.5% to 5% by weight, all weights based on the total weight of the sunscreen composition. Thus, in various embodiments, a film former is present in a composition in a weight percent amount from 0.01, 0.02, 0.03, 0.04, 0.05, 0.06, 0.07, 0.08, 0.09, 1.0, 2.0, 3.0, 4.0, 5.0, 6.0, 7.0, 8.0, 9.0, 10.0, 11.0, 12.0, 13.0, 14.0, to 15.0 percent by weight, including increments there between.

Dispersing Agents

In accordance with the various embodiments, the sunscreen composition according to the disclosure includes one or a combination of dispersing agents. In some embodiments, dispersing agents that may be used in the sunscreen composition are selected from polyester homopolymers and Modified Clay, Silica or Organic Modified Hectorite Branched-Chain Aliphatic Hydrocarbons. In some particular embodiments, the one or a combination of dispersing agents comprises one or more of Polyhydroxystearic Acid, Isohexadecane (and) Disteardimonium Hectorite (and) Propylene Carbonate, and PEG-10-Dimethicone.

In some particular embodiments, one or more of the Polyhydroxystearic Acid is present from about 0.1% to about 2%, and the Isohexadecane (and) Disteardimonium Hectorite (and) Propylene Carbonate is present from about 1% to about 4%, and the PEG-10-Dimethicone is present from about 0.05% to about 5%, each one or more present by weight, based on the total weight of the sunscreen composition.

Each one or a combination of dispersing agents is present in the sunscreen composition at a concentration from about 0.01% to 15%, in some embodiments from about 0.1% to 10%, and in some embodiments from about 0.5% to 5% by weight, all weights based on the total weight of the sunscreen composition. Thus, in various embodiments, a dispersing agent is present in a composition in a weight percent amount from 0.01, 0.02, 0.03, 0.04, 0.05, 0.06, 0.07, 0.08, 0.09, 1.0, 2.0, 3.0, 4.0, 5.0, 6.0, 7.0, 8.0, 9.0, 10.0, 11.0, 12.0, 13.0, 14.0, to 15.0 percent by weight, including increments there between.

Emulsifiers

In accordance with the various embodiments, the sunscreen composition according to the disclosure includes one or a combination of emulsifiers.

In some embodiments, emulsifiers that may be used in the sunscreen composition are selected from double fatty chain polyester copolymers and polyglyceryl fatty acid glycerol esters. In some embodiments, emulsifiers may be selected from Alkylated Sorbitol Esters and Fatty Ester: Alkylated Fatty Acid Glycol Esters and Fatty Esters; Single Fatty Chain Phosphoric Acid; Sorbitol Esters; Glycerol Ester Triglycerides; and Single Fatty Chain Fatty Acids.

In some particular embodiments, the one or a combination of emulsifiers comprises a double fatty chain polyester copolymer comprising PEG-30 Dipolyhydroxystearate, a polyglyceryl fatty acid glycerol ester comprising Polyglyceryl-4 Isostearate, or a combination thereof, each present from about 2% to about 5%, by weight, based on the total weight of the sunscreen composition.

In yet other embodiments, the one or a combination of emulsifiers comprises one or more of Polysorbate 61, PEG-40 Stearate, Potassium Cetyl Phosphate, Sorbitan Tristearate, Glyceryl Stearate, and Steric Acid, each present in the composition from about 2% to about 5%, by weight, based on the total weight of the sunscreen composition.

In some embodiments, the sunscreen composition is devoid of or will exclude or will be essentially free from emulsifiers selected from non-ionic Acrylic Acid Homopolymers; Alkyl Modified Branched Silicone, Siloxanes and Silanes Synthetic Polymers; Glycerol Esters and combinations of these. In some particular embodiments, the one or a combination of emulsifiers is not any one of Poly C10-30 Alkyl Acrylate, Lauryl PEG-9 Polydimethysiloxyethyl Dimethicone, Glyceryl Stearate (and) PEG-100 Stearate, ora combination thereof.

It will be appreciated that in embodiments wherein the emulsifier is not selected from one of the foregoing emulsifiers, the sunscreen will be devoid of or will exclude or will be essentially free from one or more of Poly C10-30 Alkyl Acrylate, Lauryl PEG-9 Polydimethysiloxyethyl Dimethicone, and Glyceryl Stearate (and) PEG-100 Stearate. These excluded emulsifiers can be associated with one or more undesirable properties that include: incompatibility with the sunscreen composition system; poor emulsion quality and instability and causes separation over time, for example at 4 weeks which separation cannot be overcome by agitations/shaking of the composition.

Each one of the one or a combination of emulsifiers is present in the sunscreen composition at a concentration from about 0.01% to 15%, in some embodiments from about 0.1% to 10%, and in some embodiments from about 0.5% to 5% by weight, all weights based on the total weight of the sunscreen composition. Thus, in various embodiments, an emulsifier is present in a composition in a weight percent amount from 0.01, 0.02, 0.03, 0.04, 0.05, 0.06, 0.07, 0.08, 0.09, 1.0, 2.0, 3.0, 4.0, 5.0, 6.0, 7.0, 8.0, 9.0, 10.0, 11.0, 12.0, 13.0, 14.0, to 15.0 percent by weight, including increments there between.

Structuring Agents

In accordance with the various embodiments, the sunscreen composition according to the disclosure includes one or a combination of structuring agents. In some particular embodiments, the one or a combination of structuring agents comprises Cetearyl Alcohol, present from about 0.5% to about 5%, Copernicia Cerifera (Carnuba) Wax/Copernicia Cerifera Cera, present from about 0.5% to about 2%, or a combination thereof, each present by weight, based on the total weight of the sunscreen composition.

In some embodiments, the one or a combination of structuring agents may compromise one or more of Behenyl Alcohol, Cetearyl Alcohol (and) Cetereth-20, Cetyl Alcohol, Stearyl Alcohol and Benzyl Alcohol, each present from about 0.5% to about 5%, by weight, based on the total weight of the sunscreen composition.

In some particular embodiments, the one or a combination of structuring agents is not Microcrystalline Wax/Cera Microcristallina.

Each one of the one or a combination of structuring agents is present in the sunscreen composition at a concentration from about 0.01% to 15%, in some embodiments from about 0.1% to 10%, and in some embodiments from about 0.5% to 5% by weight, all weights based on the total weight of the sunscreen composition. Thus, in various embodiments, a structuring agent is present in a composition in a weight percent amount from 0.01, 0.02, 0.03, 0.04, 0.05, 0.06, 0.07, 0.08, 0.09, 1.0, 2.0, 3.0, 4.0, 5.0, 6.0, 7.0, 8.0, 9.0, 10.0, 11.0, 12.0, 13.0, 14.0, to 15.0 percent by weight, including increments there between

Solvents

In accordance with various embodiments, the sunscreen composition according to the disclosure includes one or more solvents, including at least one solvent in one or both of the aqueous phase and the oil phase of the emulsion. In some embodiments, the sunscreen composition includes solvents selected from water, propanediol, ethylhexylglycerin, caprylyl glycol, C12-15 alkyl benzoate, dimethicone, caprylyl methicone, dicaprylyl carbonate, cetearyl alcohol, isohexadecane, butyloctyl salicylate, cetearyl alcohol (and) behentrimonium methosulfate, Dicaprylyl Ether, Isononyl Isononanoate, Diisopropyl Sebacate, Isododecane, Isopropyl Myristate, and combinations thereof.

More generally, in various embodiments, the solvents can include one or a combination of water, glycols, and oils/emollients that can include silicone and non-silicone compounds. In the various embodiments, the solvents can be present from about 0.05% to about 60%, or from about 0.05% to about 50%, or from about 0.1% to about 45%, or from about 1% to about 20%, or from about 1% to about 10%, or from about 1% to about 5%, or from about 2% to about 15%, or from about 3% to about 12%, or from about 4% to about 10%, or any suitable combination, sub-combination, range, or sub-range thereof by weight, based on the total weight of the sunscreen composition. One of ordinary skill in the art, however, will appreciate that other ranges are within the scope of the invention.

In some particular embodiments, the solvents can include water present in an amount that is from about 10% to about 45%. In some particular embodiments, solvents can include dimethicone and caprylyl methicone, each present from about 4% to about 10% by weight, based on the total weight of the sunscreen composition.

Thus, any one solvent or combination of solvents may be present, by weight, based on the total weight of the sunscreen composition, is from about 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 25, 30, 35, 40, 45, 50, 55 to about 60 weight percent, including increments and ranges therein and there between.

SPF Boosters

In accordance with some embodiments, the sunscreen composition according to the disclosure may comprise one or a combination of SPF boosters.

In some embodiments, the SPF booster is selected from Butyloctyl Salicylate, styrene/acrylates copolymer (such as the product sold under the tradename SUNSPHERES®), Ethylenediamine/Stearyl Dimer Dilinoleate Copolymer, Dimethicone and Acrylates/Dimethicone Copolymer, silicone polymer comprising dimethicone (and) dimethicone/vinyl dimethicone copolymer, the UVA booster Solastay, and combinations thereof. In some particular embodiments, the SPF booster comprises one or more of Butyloctyl Salicylate and styrene/acrylates copolymer.

The one or combination of SPF boosters, when present, may be at a concentration from about 0.01% to 25%, in some embodiments from about 0.1% to 13%, and in some embodiments from about 0.5% to 10, and in some embodiments from about 1% to 5% by weight, all weights based on the total weight of the sunscreen composition. Thus, in various embodiments, an SPF booster, when present, may be present in a composition in a weight percent amount from 0.01, 0.02, 0.03, 0.04, 0.05, 0.06, 0.07, 0.08, 0.09, 1.0, 2.0, 3.0, 4.0, 5.0, 6.0, 7.0, 8.0, 9.0, 10.0, 11.0, 12.0, 13.0, 14.0, 15.0, 16.0, 17.0, 18.0, 19.0, 20.0, 21.0, 22.0, 23.0, 24.0 to 25.0 percent by weight, including increments there between.

In some particular embodiments comprising an SPF booster that includes one or both of Butyloctyl Salicylate and styrene/akrylates copolymer, the SPF booster may be present from about 0.1% to about 5.0%, or from about 0.3% to about 3.0%, or from about 0.4% to about 4%, or from about 0.5% to about 2.5%, or any suitable combination, sub-combination, range, or sub-range thereof by weight, based on the total weight of the sunscreen composition. One of ordinary skill in the art, however, will appreciate that other ranges are within the scope of the invention.

Thus, the one or combination of SPF boosters that includes one or both of Butyloctyl Salicylate and styrene/akrylates copolymer, when present in the sunscreen composition, may be present from about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.5, 4.0, 4.5 to about 5.0 percent, by weight, based on the total weight of the sunscreen composition, including increments and ranges therein and there between.

Powders

In accordance with some embodiments, the sunscreen composition according to the disclosure may comprise one or a combination of sensorial feel modifying powders. In some particular embodiments, the powders may be selected from boron nitride, perlite, and aluminium starch octenyl succinate.

The one or combination of sensorial feel modifying powders, when present, may be at a concentration from about 0.05% to 15%, in some embodiments from about 0.1% to 10%, and in some embodiments from about 1% to 5% by weight, all weights based on the total weight of the sunscreen composition. Thus, in various embodiments, a powder, when present, may be present in a composition in a weight percent amount from 0.05, 0.06, 0.07, 0.08, 0.09, 1.0, 2.0, 3.0, 4.0, 5.0, 6.0, 7.0, 8.0, 9.0, 10.0, 11.0, 12.0, 13.0, 14.0, to 15.0 percent by weight, including increments there between.

Hydrating Agents

In accordance with the disclosure, in some embodiments, one or a combination hydrating agents or humectants may be present in the sunscreen composition. The hydrating agent present in the cosmetic composition, according to the disclosure, includes, but is not limited to, one or a combination of polyols, including, for example, glycerin, glycerol, glycols, such as caprylyl glycol, butylene glycol, propylene glycol, isoprene glycol, dipropylene glycol, hexylene glycol and polyethylene glycols, monoethylene glycol, diethylene glycol, triethylene glycol, diethylene glycol, hexylene glycol, glycol ethers such as monopropylene, dipropylene and tripropylene glycol alkyl(C1-C4)ethers, squalane, triacetin, sugars, such as glucose, xylitol, maltitol, sorbitol, sucrose pentaerythritol, inositol, pyrrolidone carboxylic acid, lactic acid, lithium chloride, acetamide MEA, sodium lactate, urea, dicyanamide, hyaluronic acid, aloe vera, honey, and seaweed extract.

In some embodiments, the sunscreen composition includes a hydrating agent selected from one or a combination of glycerin present at about 4% and caprylyl glycol present at about 1%.

In accordance with the various embodiments, the amount of hydrating agent present in the sunscreen composition can range from about 1% to about 25%, or from about 2% to about 20%, or from about 3% to about 5% or any suitable combination, sub-combination, range, or sub-range thereof by weight, based on the total weight of the sunscreen composition. One of ordinary skill in the art, however, will appreciate that other ranges are within the scope of the invention.

Thus, any one of or a combination of hydrating agent may be present, by weight, based on the total weight of the sunscreen composition, is from about 1, 2, 3, 4, 5, 6, 7, 8, 9, 10, 11, 12, 13, 14, 15, 16, 17, 18, 19, 20, 21, 22, 23, 24, to about 25 weight percent, including increments and ranges therein and there between.

Actives

The sunscreen compositions can also comprise one or a combination of actives used in the cosmetics field which does not affect the properties of the sunscreen compositions according to the invention, such as fragrances, pearlescent agents, silica, preservatives, proteins, protein hydrolysates, sodium hyaluronate, vitamins, panthenol, silicones, odor absorbers and coloring materials; anti-microbial components, including, but not limited to, phenoxyethanol, chlorphenesin, capryloyl glycol and sodium salicylate; essential oils selected from the group consisting of sunflower oil, sesame oil, peppermint oil, macadamia nut oil, tea tree oil, evening primrose oil, sage oil, rosemary oil, coriander oil, thyme oil, pimento berries oil, rose oil, anise oil, balsam oil, bergamot oil, rosewood oil, cedar oil, chamomile oil, sage oil, clary sage oil, clove oil, cypress oil, eucalyptus oil, fennel oil, sea fennel oil, frankincense oil, geranium oil, ginger oil, grapefruit oil, jasmine oil, juniper oil, lavender oil, lemon oil, lemongrass oil, lime oil, mandarin oil, marjoram oil, myrrh oil, neroli oil, orange oil, patchouli oil, pepper oil, black pepper oil, petitgrain oil, pine oil, rose otto oil, rosemary oil, sandalwood oil, spearmint oil, spikenard oil, vetiver oil, wintergreen oil, and ylang ylang; fruit extracts, for example Pyrus Malus (Apple) Fruit Extract, and Aloe Barbadensis Leaf Juice Powder; citric acid, sodium chloride; neutralizing or pH-adjusting agents (e.g., triethylamine (TEA) and sodium hydroxide), and combinations thereof.

Although the optional actives are given as examples, it will be appreciated that other optional components compatible with cosmetic applications known in the art may be used. In some particular embodiments, the sunscreen composition may include antimicrobials comprising one or a combination of capryloyl glycol and phenoxyethanol.

In some particular embodiments, the sunscreen composition may include actives comprising one or a combination of Disodium EDTA, Sodium Hyaluronate, Sodium Lauroyl Lactylate (And) Ceramide NP (And) Ceramide AP (And) Phytosphingosine (And) Cholesterol (And) Xanthan Gum (And) Carbomer (And) Ceramide EOP, Trisodium Ethylenediamine Disuccinate, panthenol, tocopherol, niacinamide, and Magnesium Sulfate.

In some particular embodiments, the sunscreen composition may include actives comprising one or a combination of thickeners selected from Hydroxypropyl Methylcellulose, Ammonium Polyacryloyldimethyl Taurate, and Ethylene/Acrylic Acid Copolymer.

In accordance with the various embodiments, the amount of each one or a combination of actives, when present in the sunscreen composition can be present in a range from about 0.001% to about 20%, by weight, or from about 0.005% to about 0.01%, or from about 0.01% to about 0.1%, or from about 0.15% to about 5%, or from about 0.40% to about 4%, or from about 0.5% to about 2.5% by weight, or from about 1% to about 2%, or any suitable combination, sub-combination, range, or sub-range thereof by weight, based on the total weight of the sunscreen composition. And in some embodiments, a combination of actives present in the sunscreen composition can be present in a range from about 0.001% to about 20%.

Thus, any one or a combination of actives, when present, may be present, by weight, based on the total weight of the sunscreen composition, each one or the combination present from about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.10, 0.20, 0.30, 0.40, 0.50, 0.60, 0.70, 0.80, 0.90, 1.0, 2, 3, 4, 5, 6, 7, 8, 9, 10, 11, 12, 13, 14, 15, 16, 17, 18, 19 to about 20 weight percent, including increments and ranges therein and there between.

### EXAMPLES

### Example 1: Raw Materials

The raw materials as used in the inventive compositions are set forth in Table 1, below.

TABLE 1
Raw Materials with RM other than at 100%
INGREDIENT

ZINC OXIDE (and) TRIETHOXYCAPRYLYLSILANE, 97% RM
DIMETHICONE (and) ACRYLATES/DIMETHICONE COPOLYMER
(60%/40%)
ISOHEXADECANE (and) DISTEARDIMONIUM HECTORITE (and)
PROPYLENE CARBONATE (87%/10%/3%)
SUNSPHERES (86% RM)

### Example 2: Inventive Compositions

The inventive compositions as set forth in Table 2, below, include

TABLE 2
Inventive Compositions

| | INV | INV | INV | INV | INV |
|---|---|---|---|---|---|
| INCI US | 1 | 2 | 3 | 4 | 5 |
| BUTYLOCTYL SALICYLATE | 3. | 3. | 3. | 3. | 3. |
| ETHYLHEXYL SALICYLATE | 5. | 5. | 5. | 5. | 5. |
| POLYHYDROXYSTEARIC | 2. | 2. | 2. | 2. | 2. |
| ACID | | | | | |
| CETEARYL ALCOHOL | 3. | 3. | 3. | 3. | 3. |
| OCTOCRYLENE | 10. | 10. | 10. | 10. | 10. |
| COPERNICIA CERIFERA | 1. | 1. | 1. | 1. | 1. |
| (CARNAUBA) WAX | | | | | |
| POLYGLYCERYL-4 | 3. | 3.5 | 3. | 3.5 | 3. |
| ISOSTEARATE | | | | | |
| ISOHEXADECANE (and) | 4. | 4. | 4. | 4. | 4. |
| DISTEARDIMONIUM | | | | | |
| HECTORITE (and) | | | | | |
| PROPYLENE CARBONATE | | | | | |
| ZINC OXIDE (and) | 15.39 | 12.31 | 12.31 | 12.31 | 12.31 |
| TRIETHOXYCAPRYLYL- | | | | | |
| SILANE | | | | | |
| PEG-30 DIPOLYHYDROXY- | 3. | 3.5 | 3. | 3.5 | 3. |
| STEARATE | | | | | |
| WATER | 29.81 | 19.13 | 21.63 | 18.13 | 20.63 |
| TOCOPHEROL | 0.1 | 0.5 | 0.5 | 0.5 | 0.5 |
| HOMOSALATE | | 15. | 15. | 15. | 15. |
| DIMETHICONE (and) | | 2. | | 2. | |
| ACRYLATES/DIMETHICONE | | | | | |
| COPOLYMER | | | | | |
| MAGNESIUM SULFATE | | 0.1 | 0.1 | 0.1 | 0.1 |
| POLYESTER-5 | | 2. | 2. | 2. | 2. |
| SOLVENTS (polar oils, spreading | 16 | | | | |
| solvents, dispersants) | | | | | |
| SILICONES (solvents, anti- | | 5 | 8 | 5 | 8 |
| foaming agents, spreading | | | | | |
| agents) | | | | | |
| ACTIVES | | 1.56 | 1.56 | 1.56 | 1.56 |
| PRESERVATIVES | 0.7 | 0.7 | 0.7 | 0.7 | 0.7 |
| CO-PRESERVATIVES | 0.35 | 0.35 | 0.35 | 0.35 | 0.35 |
| HUMECTANT | 0.5 | 0.5 | 0.5 | 0.5 | 0.5 |
| CHELATOR | 0.1 | 0.3 | 0.3 | 0.3 | 0.3 |
| POLYMER | 0.55 | 0.055 | 0.55 | 0.55 | 0.55 |
| SENSORY: AESTHETIC | 1 | 2 | 1 | 3 | 2 |
| MODIFIERS, MATTIFYING | | | | | |
| AGENTS, SOFTENING AGENTS | | | | | |

### Example 3: Demonstration of WR and Critical Wavelength Results with Inventive and Comparative Compositions

Comparative Sunscreen compositions and inventive compositions were evaluated for performance based on properties that included stability, sensorial/textural features, critical wavelength and water resistance (WR) test performance. Results are shown in Table 3, for which the following key is applicable.

Key:

Base**=formula defined by common emulsifiers, dispersing agents, structuring agents and film formers, wherein: BASE A includes: Water-in-oil emulsion with PEG-30 Dipolyhydroxystearate and Polygylceryl-4 Isostearate as emulsifiers; Polyhydroxystearic Acid and Isohexadecane (and) Disteardimonium Hectorice (and) Propylene Carbonate as dispersing agents; Cetearyl Alcohol and Carnuba Wax as structuring agents; Polyester-5 and Dimethicone (and) Acrylates/Dimethicone Copolymer as film formers and wherein BASE B includes; Water-in-oil emulsion with PEG-30 Dipolyhydroxystearate, Polygylceryl-4 Isostearate, and Lauryl PEG-9 Polydimethylsiloxyethyl Dimethicone as emulsifiers; Polyhydroxystearic Acid and Isohexadecane (and) Disteardimonium Hectorice (and) Propylene Carbonate as dispersing agents; Hydrogenated Jojoba Oil and Poly C10-30 Alkyl Acrylate as structuring agents; no defined film formers and wherein BASE C includes; Oil-in-water emulsion with Polysorbate 61, PEG-40 Stearate, Potassium Cetyl Phosphate, and Sorbitan Tristearate as emulsifiers; Isohexadecane (and) Disteardimonium Hectorice (and) Propylene Carbonate as dispersing agent; no defined structuring agent; and Ethylenediaminestearyl dimer dilnoleate copolymer as film former and wherein BASE D includes; Glyceryl Stearate (and) PEG-100 Stearate, and Potassium Cetyl Phosphate as emulsifiers; Isohexadecane (and) Disteardimonium Hectorice (and) Propylene Carbonate as dispersing agent; Acrylates/C10-30 Alkyl Acrylate Crosspolymer, and Poly C10-30 Alkyl Acrylate as structuring agents; Dimethicone (and) Acrylates/Dimethicone Copolymer as film formers. *Invalid means that the SPF could not be effectively measured as defined by FDA 2011 SPF testing Method and/or on the tested subject.

SPF Initial=SPF measured before WR test (water immersion of treated substrate).

SPF #=SPF after WR Test Can WR 80 mins be clarified? It is more difficult to achieve than WR 40 mins.

Critical WL=Critical Wavelength, where composition fails if the measured WL is <370 nm.

TABLE 3
Performance Results with various COMP and INV compositions

| TESTED | | | | | | |
|---|---|---|---|---|---|---|
| COMP | | | SPF | | Critical | |
| (Base)** | UV Filters | Film former | Initial | SPF # | WL | RESULTS |
| BASE A; | 12% ZnO | NONE | 50 | 34.5* | N/A | Failed WR |
| COMP 1 | 15% Homosalate | | | INVALID | | |
| | 5% Octisalate | | | | | |
| | 10% Octocrylene | | | | | |
| BASE A; | 20% ZnO | NONE | 54.5 | 34.5* | N/A | Failed WR |
| COMP 2 | 15% Homosalate | | | INVALID | | |
| | 5% Octisalate | | | | | |
| | 10% Octocrylene | | | | | |
| BASE A; | 15% ZnO | NONE | 43.5 | 34.5* | N/A | Failed WR |
| COMP 3 | 15% Homosalate | | | INVALID | | |
| | 5% Octisalate | | | | | |
| | 10% Octocrylene | | | | | |
| BASE A; | 12% ZnO | Polyurethane-35 | N/A | N/A | N/A | Failed due |
| COMP 4 | 15% Homosalate | | | | | to residue |
| | 5% Octisalate | | | | | |
| | 10% Octocrylene | | | | | |
| BASE A; | 12% ZnO | Trimethylsiloxysilicate | N/A | N/A | N/A | Failed |
| COMP 5 | 15% Homosalate | | | | | Stability |
| | 5% Octisalate | | | | | |
| | 10% Octocrylene | | | | | |
| BASE A; | 12% ZnO | ETHYLENE DIAMINE/ | N/A | N/A | N/A | Failed |
| COMP 6 | 15% Homosalate | STEARYL DIMER | | | | Stability |
| | 5% Octisalate | DILINOLEATE | | | | |
| | 10% Octocrylene | COPOLYMER | | | | |
| BASE A; | 12% ZnO | Polyester-5 | 53.1 | 49.35 | 379.58 | Met WR; |
| INV 1 | 15% Homosalate | Dimethicone | | | | exceeded |
| | 5% Octisalate | (and) Acrylates/ | | | | Critical |
| | 10% Octocrylene | Dimethicone | | | | WL; Stable; |
| | | copolymer | | | | no residue |
| BASE A; | 12% ZnO | Polyester-5 | 41.3 | 36.4 | 373.67 | Met WR; |
| INV 2 | 15% Homosalate | | | | | exceeded |
| | 5% Octisalate | | | | | Critical |
| | 10% Octocrylene | | | | | WL; stable; |
| | | | | | | no residue |
| BASE A; | 15% ZnO | Polyester-5 | 50 | *INVALID | N/A | Failed |
| COMP 7 | 15% Homosalate | Dimethicone | | | | stability |
| | 5% Octisalate | (and) Acrylates/ | | | | |
| | 10% Octocrylene | Dimethicone | | | | |
| | | copolymer | | | | |
| BASE A; | 12% ZnO | Polyester-5 | 28.7 | *INVALID | N/A | Failed WR |
| COMP 8 | 15% Homosalate | | | | | |
| | 5% Octisalate | | | | | |
| | 10% Octocrylene | | | | | |
| BASE A; | 12% ZnO | Polyester-5 | 50.5 | 47.83* | N/A | Failed WR |
| COMP 9 | 15% Homosalate | Dimethicone | | INVALID | | |
| | 5% Octisalate | (and) Acrylates/ | | | | |
| | 10% Octocrylene | Dimethicone | | | | |
| | | copolymer | | | | |
| BASE A; | 12% ZnO | Polyester-5 | 32.9 | 30.9 | N/A | Failed |
| COMP 10 | 15% Homosalate | | | | | textural |
| | 5% Octisalate | | | | | sensorial |
| | 10% Octocrylene | | | | | profile, |
| | | | | | | draggy, |
| | | | | | | difficult to |
| | | | | | | spread |
| BASE A; | 12% ZnO | Polyester-5 | 54.75 | 43.5* | N/A | Failed WR; |
| COMP 11 | 15% Homosalate | Dimethicone | | INVALID | | Failed |
| | 5% Octisalate | (and) Acrylates/ | | | | textural |
| | 10% Octocrylene | Dimethicone | | | | sensorial |
| | | copolymer | | | | profile, |
| | | | | | | draggy, |
| | | | | | | difficult to |
| | | | | | | spread |
| BASE A; | 15% ZnO | Polyester-5 | 28.8 | 27.3 | N/A | Failed WR; |
| COMP 12 | 15% Homosalate | Dimethicone | | | | Failed |
| | 5% Octisalate | (and) Acrylates/ | | | | textural |
| | 10% Octocrylene | Dimethicone | | | | sensorial |
| | | copolymer | | | | profile, |
| | | | | | | draggy, |
| | | | | | | difficult to |
| | | | | | | spread |
| BASE A; | 12% ZnO | Polyester-5 | 34.7 | 30.2 | N/A | Failed |
| COMP 13 | 15% Homosalate | Dimethicone | | | | textural |
| | 5% Octisalate | (and) Acrylates/ | | | | sensorial |
| | 10% Octocrylene | Dimethicone | | | | profile, |
| | | copolymer | | | | draggy, |
| | | | | | | difficult to |
| | | | | | | spread |
| BASE A; | 12% ZnO | Polyester-5 | 61.55 | 51 | optimized | Improved |
| INV 6 | 15% Homosalate | | | | stability, | stability |
| | 5% Octisalate | | | | added | and sensorial |
| | 10% Octocrylene | | | | sensory | feel with |
| | | | | | powder- | additional |
| | | | | | | powder |
| BASE A; | 12% ZnO | Polyester-5 | 41.85 | 44 | optimized | Improved |
| INV 7 | 15% Homosalate | Dimethicone | | | stability, | stability |
| | 5% Octisalate | (and) Acrylates/ | | | added | and sensorial |
| | 10% Octocrylene | Dimethicone | | | sensory | feel with |
| | | copolymer | | | powder | additional |
| | | | | | | powder |
| BASE B; | 5% Zinc Oxide | NONE | N/A | N/A | 365.2 | Failed |
| COMP 14 | 10% Homosalate | | | | | Critical WL |
| | 5% Octisalate | | | | | |
| | 10% Octocrylene | | | | | |
| BASE B; | 5% Zinc Oxide | NONE | 37.05 | *INVALID | N/A | Failed WR |
| COMP 15 | 15% Homosalate | | | | | and stability |
| | 5% Octisalate | | | | | |
| BASE B; | 5% Zinc Oxide | NONE | 57.75 | 36* | N/A | Failed WR |
| COMP 16 | 15% Homosalate | | | INVALID | | and stability |
| | 5% Octisalate | | | | | |
| | 10% Octocrylene | | | | | |
| BASE B; | 10% Zinc Oxide | ETHYLENE DIAMINE/ | 50.33 | 43.5 | 373.15 | Failed WR |
| COMP 17 | 15% Homosalate | STEARYL DIMER | | | | |
| | 5% Octisalate | DILINOLEATE | | | | |
| | 10% Octocrylene | COPOLYMER | | | | |
| | | Trimethylsiloxysilicate | | | | |
| BASE B; | 12% Zinc Oxide | ETHYLENE DIAMINE/ | *INVALID | *INVALID | 373.58 | Failed WR |
| COMP 18 | 15% Homosalate | STEARYL DIMER | | | | |
| 202158949 | 5% Octisalate | DILINOLEATE | | | | |
| | 10% Octocrylene | COPOLYMER | | | | |
| | | Trimethylsiloxysilicate | | | | |
| BASE B; | 12% Zinc Oxide | ETHYLENE DIAMINE/ | 34.2 | 32.3 | N/A | Failed WR |
| COMP 19 | 15% Homosalate | STEARYL DIMER | | | | and stability |
| 202158953 | 5% Octisalate | DILINOLEATE | | | | |
| | 10% Octocrylene | COPOLYMER | | | | |
| | | Trimethylsiloxysilicate | | | | |
| BASE C; | 5% ZnO | NONE | 39.6 | *INVALID | N/A | Failed WR |
| COMP 20 | 15% Homosalate | | | | | |
| 20215964 | 5% Octisalate | | | | | |
| | 7% Octocrylene | | | | | |
| BASE D; | 1.96% ZnO | NONE | N/A | N/A | N/A | Failed |
| COMP 21 | 10% Homosalate | | | | | stability; |
| 8553392 | 5% Octisalate | | | | | poor emulsion |
| | 10% Octocrylene | | | | | quality and |
| | | | | | | macro |
| | | | | | | appearance |
| | | | | | | on day 0 |
| BASE D; | 1.96% ZnO | NONE | N/A | N/A | N/A | Failed |
| COMP 22 | 10% Homosalate | | | | | stability; |
| 8553391 | 5% Octisalate | | | | | poor emulsion |
| | 10% Octocrylene | | | | | quality and |
| | | | | | | macro |
| | | | | | | appearance |
| | | | | | | on day 0 |

While the disclosure has been described with reference to a preferred embodiment, it will be understood by those skilled in the art that various changes may be made, and equivalents may be substituted for elements thereof without departing from the scope of the disclosure. In addition, many modifications may be made to adapt a particular situation or material to the teachings of the disclosure without departing from the essential scope thereof. Therefore, it is intended that the disclosure is not limited to the particular embodiment disclosed as the best mode contemplated for carrying out this disclosure, but that the disclosure will include all embodiments falling within the scope of the appended claims.

The articles “a” and “an,” as used herein, mean one or more when applied to any feature in embodiments of the present disclosure described in the specification and claims. The use of “a” and “an” does not limit the meaning to a single feature unless such a limit is specifically stated. The article “the” preceding singular or plural nouns or noun phrases denotes a particular specified feature or particular specified features and may have a singular or plural connotation depending upon the context in which it is used. The adjective “any” means one, some, or all indiscriminately of whatever quantity.

“At least one” or “one or more” or “one or a combination of” or “more than one” as used herein, means that there may be one, two, three or more and thus includes individual components as well as mixtures/combinations.

The transitional terms “comprising”, “consisting essentially of” and “consisting of”, when used in the appended claims, in original and amended form, define the claim scope with respect to what unrecited additional claim elements or steps, if any, are excluded from the scope of the claim(s). The term “comprising” is intended to be inclusive or open-ended and does not exclude any additional, unrecited element, method, step or material. The term “consisting of” excludes any element, step or material other than those specified in the claim and, in the latter instance, impurities ordinarily associated with the specified material(s). The term “consisting essentially of” limits the scope of a claim to the specified elements, steps or material(s) and those that do not materially affect the basic and novel characteristic(s) of the claimed disclosure. All materials and methods described herein that embody the present disclosure can, in alternate embodiments, be more specifically defined by any of the transitional terms “comprising,” “consisting essentially of,” and “consisting of.”

The terms “free” and “devoid” indicates that no reliably measurable excluded material is present in the sunscreen composition, typically 0% by weight, based on the total weight of the sunscreen composition. The term “essentially free” means that, while it prefers that no excluded material is present in the sunscreen composition, it is possible to have very small amounts of the excluded material in the sunscreen composition of the invention, provided that these amounts do not materially affect the advantageous properties of the sunscreen composition. In particular, “essentially free” means that excluded material can be present in the sunscreen composition at an amount of less than about 0.1% by weight, based on the total weight of the sunscreen composition.

Other than in the operating examples, or where otherwise indicated, all numbers expressing quantities of ingredients and/or reaction conditions are to be understood as being modified in all instances by the term “about,” meaning within 10% of the indicated number (e.g. “about 10%” means 9%-11% and “about 2%” means 1.8%-2.2%).

All percentages and ratios are calculated by weight unless otherwise indicated. All percentages are calculated based on the total composition unless otherwise indicated. Generally, unless otherwise expressly stated herein, “weight” or “amount” as used herein with respect to the percent amount of an ingredient refers to the amount of the raw material comprising the ingredient, wherein the raw material may be described herein to comprise less than and up to 100% activity of the ingredient. Therefore, weight percent of an active in a composition is represented as the amount of raw material containing the active that is used and may or may not reflect the final percentage of the active, wherein the final percentage of the active is dependent on the weight percent of active in the raw material.

All ranges and amounts given herein are intended to include subranges and amounts using any disclosed point as an end point. Thus, a range of “1% to 10%, such as 2% to 8%, such as 3% to 5%,” is intended to encompass ranges of “1% to 8%,” “1% to 5%,” “2% to 10%,” and so on. All numbers, amounts, ranges, etc., are intended to be modified by the term “about,” whether or not so expressly stated. Similarly, a range given of “about 1% to 10%” is intended to have the term “about” modifying both the 1% and the 10% endpoints. Further, it is understood that when an amount of a component is given, it is intended to signify the amount of the active material unless otherwise specifically stated.

Notwithstanding that the numerical ranges and parameters setting forth the broad scope of the disclosure are approximations, unless otherwise indicated the numerical values set forth in the specific examples are reported as precisely as possible. Any numerical value, however, inherently contains certain errors necessarily resulting from the standard deviation found in their respective testing measurements. The example that follows serves to illustrate embodiments of the present disclosure without, however, being limiting in nature.

All publications and patent applications cited in this specification are herein incorporated by reference, and for any and all purposes, as if each individual publication or patent application were specifically and individually indicated to be incorporated by reference. In the event of an inconsistency between the present disclosure and any publications or patent application incorporated herein by reference, the present disclosure controls.

What is claimed is:

1. A sunscreen composition, comprising:
a water-in-oil emulsion of aqueous and oil phases, comprising the following components:
(i) at least one mineral UV filter, present from about 10% to about 15%;
(ii) a blend of organic UV filters, present from about 15% to about 45%;
(iii) at least one film former, present from about 2% to about 5%;
(iv) one or a combination of dispersing agents;
(v) one or a combination of emulsifiers;
(vi) one or a combination of structuring agents; and
(vii) one or a combination of solvents,
wherein the components are present by weight, based on the total weight of the sunscreen composition, and
wherein the sunscreen composition confers water-resistant broad-spectrum UV protection, is non-whitening, and demonstrates acceptable sensorial properties and emulsion structural stability through a range of temperatures.

2. The sunscreen composition according to claim 1, wherein the sunscreen composition is free from one or both of oxybenzone and octinoxate organic UV filters.

3. The sunscreen composition according to claim 1, wherein the inorganic UV filters include zinc oxide and wherein the organic UV filters include each of homosalate, octisalate and octocrylene.

4. The sunscreen composition according to claim 1, wherein the weight ratio of organic:inorganic/mineral UV filters is in the range from about 2:1 to about 4:1, based on the total weight of the sunscreen composition.

5. The sunscreen composition according to claim 1, wherein the inorganic UV filters include zinc oxide, present in an amount in the range from about 10% to about 14%, and wherein the organic UV filters include homosalate, present in an amount in the range from about 12% to about 20%, octisalate, present in an amount in the range from about 1% to about 7%, and octocrylene, present in an amount in the range from about 7% to about 15%, each present by weight, based on the total weight of the sunscreen composition.

6. The sunscreen composition according to claim 1, wherein the inorganic UV filters include zinc oxide, present in an amount in the range from about 10% to about 12%, and wherein the organic UV filters include homosalate, present at about 15%, octisalate, present at about 5%, and octocrylene, present at about 10%, each present, by weight, based on the total weight of the sunscreen composition.

7. The sunscreen composition according to claim 1, wherein the at least one film former comprises one or more of polyester-5, present from about 1% to about 3%, and dimethicone acrylates copolymer, present from about 2% to about 5%, and a combination thereof, each by weight, based on the total weight of the sunscreen composition.

8. The sunscreen composition according to claim 1, wherein the at least one film former is not any one of Polyurethane-35, Trimethylsiloxysilicate, Ethylenediamine/Stearyl Dimer Dilinoleate Copolymer, or a combination thereof.

9. The sunscreen composition according to claim 1, wherein the one or a combination of dispersing agents comprises Polyhydroxystearic Acid, Isohexadecane (and) Disteardimonium Hectorite (and) Propylene Carbonate, or a combination thereof.

10. The sunscreen composition according to claim 9, wherein one or more of the Polyhydroxystearic Acid is present from about 0.1% to about 2%, by weight, based on the total weight of the sunscreen composition, and the Isohexadecane (and) Disteardimonium Hectorite (and) Propylene Carbonate is present from about 1% to about 4%, by weight, based on the total weight of the sunscreen composition.

11. The sunscreen composition according to claim 1, wherein the one or a combination of emulsifiers comprises PEG-30 Dipolyhydroxystearate, Polyglyceryl-4 Isostearate, or a combination thereof, present from about 2% to about 5%, by weight, based on the total weight of the sunscreen composition.

12. The sunscreen composition according to claim 1, wherein the one or a combination of emulsifiers is not any one of Poly C10-30 Alkyl Acrylate, Lauryl PEG-9 Polydimethysiloxyethyl Dimethicone, Glyceryl Stearate (and) PEG-100 Stearate or a combination thereof.

13. The sunscreen composition according to claim 1, wherein the one or a combination of structuring agents comprises Cetearyl Alcohol, present from about 0.5% to about 5%, Copernicia Cerifera (Carnuba) Wax/Copernicia Cerifera Cera, present from about 0.5% to about 2%, or a combination thereof, each by weight, based on the total weight of the sunscreen composition.

14. The sunscreen composition according to claim 13, wherein the one or a combination of structuring agents is not Microcrystalline Wax/Cera Microcristallina.

15. The sunscreen composition according to claim 1, wherein the combination of solvents comprises one or more esters, ethers, alkanes, silicones, and combinations thereof.

16. The sunscreen composition according to claim 1, wherein the ratio of solvent to structuring agent is in the range from about 3:1 to about 8:1.

17. The sunscreen composition according to claim 1, further comprising one or more SPF boosters, one or more sensorial fatty compounds, one or more feel modifying powders, or one or more actives.

18. The sunscreen composition according to claim 1, the sunscreen composition having an SPF in a range from about 30 to about 55.

19. A sunscreen composition, comprising:
a water-in-oil emulsion of aqueous and oil phases, comprising the components:
(i) at least one mineral UV filter comprising ZnO, present at about 12%;
(ii) a blend of organic UV filters comprising homosalate, present at about 15%, octisalate, present at about 5%, and octocrylene, present at about 10%;
(vii) at least one film former comprising one or more of polyester-5 and dimethicone acrylates copolymer, present from about 2% to about 5%;
(iii) a combination of dispersing agents comprising polyhydroxystearic acid, and isohexadecane (and) disteardimonium hectorite (and) propylene carbonate;
(iv) a combination of emulsifiers comprising PEG-30 di polyhydroxystearate and polyglyceryl-4 isostearate;
(v) one or a combination of structuring agents;
(viii) one or a combination of solvents; and
(ix) one or more SPF boosters, one or more sensorial fatty compounds or feel modifying powders, or one or more actives,
wherein the components are present by weight, based on the total weight of the sunscreen composition.

20. A sprayable sunscreen composition, comprising:
a water-in-oil emulsion of aqueous and oil phases, comprising the following components:
(i) at least one mineral UV filter, present from about 10% to about 15%;
(ii) a blend of organic UV filters, present from about 15% to about 45%;
(iii) at least one film former, present from about 2% to about 5%;
(iv) one or a combination of dispersing agents;
(v) one or a combination of emulsifiers; and
(vi) one or a combination of solvents,
wherein the components are present by weight, based on the total weight of the sunscreen composition, and
wherein the sunscreen composition is sprayable and confers water-resistant broad-spectrum UV protection, is non-whitening, and demonstrates acceptable sensorial properties and emulsion structural stability through a range of temperatures.
